(19) United States
(12) Patent Application Publication
(10) Pub. No.: US 2005/0080389 A1
(43) Pub. Date: Apr. 14, 2005

(54) Absorbent articles having increased absorbency of complex fluids

(75) Inventors: Alice Yvonne Romans-Hess, Pinehurst, NC (US); Jason Matthew English, Appleton, WI (US); Jian Qin, Appleton, WI (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Kimberly-Clark Worldwide, Inc.

(21) Appl. No.: 10/684,181

(22) Filed: Oct. 10, 2003

(51) Int. Cl. A61F 13/15; B32B 5/16; A61F 13/20; B32B 27/02

(52) U.S. Cl. 604/367; 428/402; 442/417

(57) ABSTRACT

Absorbent articles comprising superabsorbent particles having an increased surface area to volume ratio are disclosed. The superabsorbent particles comprising the absorbent articles are in flake-like form, and have an average surface area to volume ratio of at least about 0.05 μm⁻¹. In one specific embodiment, the high surface area to volume superabsorbent particles have an average thickness of no more than about 30 micrometers.

### BACKGROUND OF THE INVENTION

The present invention relates to absorbent articles comprising superabsorbent particles capable of quickly absorbing an increased amount of complex fluids, such as plasma, blood, menses, mucus, or liquid bowel movement, as compared to conventional absorbent articles comprising conventional spherical-type superabsorbent particles. More particularly, the present invention relates to feminine napkins and other absorbent devices, which comprise at least some flake-like superabsorbent particles having an average surface area to volume ratio of at least about 0.05 μm⁻¹, and which have increased absorbency characteristics for complex fluids as compared to conventional devices.

The use of superabsorbent particles (also commonly referred to as superabsorbent polymers and superabsorbent materials), such as ionic superabsorbent polymers, is standard throughout the absorbent products industry. The absorbent core or structure of many absorbent articles, such as diapers, feminine napkins, training pants, and incontinence products, is typically comprised of one or more layers of cellulose fluff pulp, intermixed with superabsorbent particles. Other layers are often used in combination with the absorbent core depending upon the intended application of the product. Superabsorbent particles may also be located in areas other than, or in addition to, the absorbent core.

Ionic superabsorbent polymers, also commonly referred to as ionic hydrogels or ionic hydrocolloids, are typically cross-linked ionic polymers that are able to absorb an amount of 0.9 weight percent sodium chloride saline or urine equal to at least ten times their dry weight and retain the absorbed fluid under a moderate external pressure. Ionic superabsorbent polymers can be anionic in nature (e.g., acrylate based or sulfonate based), or can be cationic in nature (e.g., a partly neutralized polyamine), and as such can either have positive or negative charges along the backbone of the polymer structure. Ionic superabsorbent polymers are electrically charged in solution because various groups attached to the polymer chain easily become ionic. Examples of groups that can become electrically charged in ionic superabsorbent polymers include carboxyl groups and amino groups.

Current commercially available spherical-like superabsorbent polymers are generally prepared by mixing an ethylenically unsaturated monomer, a neutralization agent, a crosslinking agent, and an initiator with water to form a monomer solution, which is subsequently heated to form a hydrogel solution. The water-containing hydrogel is then typically cut, dried and finally ground into particles having a particle size range of from about 100 micrometers to about 850 micrometers and an average size of about 300 to about 400 micrometers. This procedure produces spherical-like or chunk-like superabsorbent particles.

Some superabsorbent polymers may be manufactured utilizing pre-polymers in place of monomers. When pre-polymers are utilized, superabsorbent polymers are typically prepared by dissolving a suitable polymer in water and adding a crosslinking agent into the aqueous polymeric solution while vigorously stirring to produce a uniform solution. The uniform solution is then dried and ground into the desired particle size distribution prior to heating the particles to induce crosslinking to insolubilize the resulting superabsorbent polymers. Regardless of whether superabsorbent particles are prepared with monomers or pre-polymers, they are typically considered granular in nature (spherical-like or cubical-like) with nearly equal size in length, width, and thickness in the x, y, and z directions.

Liquid absorbed by a superabsorbent polymer is taken directly into the molecular structure itself, and is not simply contained in pores or openings in the material from which it could be easily expressed by the application of pressure. The typical commercially available ionic superabsorbent polymers are generally crosslinked polyacrylates such as poly(acrylic acid) or acrylic acid grafted onto starch. The carboxyl functionality on the polymer backbone is partially neutralized using a basic compound such as sodium or potassium hydroxide. Some ionic superabsorbent polymers are formed by graft polymerizing acrylonitrile onto gelatinized starch followed by hydrolysis of the polyacrylonitrile to poly(acrylic acid-co-acrylamide).

Although ionic superabsorbent polymers have the ability to absorb many times their weight of pure water, when contacted with aqueous salt solutions, their ability to effectively absorb liquid is generally reduced by a factor of five, or even more, depending upon the ionic strength of the salt solution. The degradation of absorption capacity suffered by ionic polymers in salt solutions is believed to be due to a collapse of the counterion atmosphere surrounding the ionic backbone of the polymer chains. The counterion atmosphere is made up of ions of opposite charge to the charges along the backbone of the ionic polymer. These counterions are present in the salt solution (e.g., sodium or potassium cations surrounding the carboxylate anions distributed along the backbone of a polyacrylate anionic polymer). As the concentration of ions in the salt solution contacting the superabsorbent ionic polymer increases, the ion concentration gradient in the liquid phase from the exterior to the interior of the polymer begins to decrease and the counterion atmosphere thickness, typically referred to as the Debye thickness, can be reduced from about 20 nanometers (in pure water) to about 1 nanometer or less. When the counterion atmosphere is highly extended (i.e., not significantly collapsed around the polymer backbone as when pure water is absorbed), the counterions are much more osmotically active and therefore promote a much higher degree of liquid absorbency. However, when the ion concentration in the absorbed liquid increases, the counterion atmosphere collapses and absorption capacity of the polymer is diminished.

Additionally, complex bodily fluids such as plasma, blood, menses, mucus, and liquid bowel movement are particularly difficult to effectively absorb into superabsorbent products due to the viscosity and complex nature of the fluids. For example, plasma, blood and menses components, including red cells, white cells, soluble proteins, cellular debris and mucus, slow down the absorption of these fluids by conventional superabsorbents. Because these fluids comprise many complex components, and are generally very thick and gel-like, absorption into a superabsorbent polymer is difficult. The slower initial uptake rate of the fluid into the superabsorbent polymer can result in a lower final capacity if gel blocking occurs before the superabsorbent polymer is fully swollen. Also, because of the volume and insult rate of blood products, and notably menses, superabsorbent polymers may not be fully swollen in blood under normal wear conditions. This can result in wasted superabsorbent capacity and increased cost.

Traditionally, efforts to increase the capacity of superabsorbent materials for blood products, such as menses, and liquid bowel movements, have centered around increasing the speed of fluid uptake and potential capacity of the superabsorbent polymer. For example, one way of increasing the speed of fluid uptake that has been tried is to decrease the size of the superabsorbent particles to decrease the thickness of the polymer that must be penetrated by the fluid to fully swell the entire particle. Although this approach has achieved some success in the absorption of blood and menses, it has been associated with several undesirable processing and consumer use concerns. Specifically, the use of very small spherical-like superabsorbent particles has resulted in dusting and conveying difficulties in manufacture and the failure to retain the smaller particles within the consumer product during manufacturing and packaging, as well as during use by the consumer.

Another traditional means of attempting to increase the capacity of superabsorbent particles for blood products and menses has included increasing the porosity internal to the structure of the superabsorbent particle. The resultant lower bulk density of the polymer may present processing difficulties in the area of polymer conveying and feeding, as well as increased manufacturing and transportation costs and changes in other polymer properties.

Another means of attempting to increase the capacity of superabsorbent particles for blood products and menses has included the use of a fibrous superabsorbent material, such as a staple fiber or filament. Although the fibrous superabsorbent material may have enticing thickness and average surface area to volume ratios, it does not have sufficient rigidity and requires special equipment on the manufacturing lines in order to properly feed the material into absorbent products, such as diapers. This can significantly increase manufacturing costs.

As is evident from the foregoing, a need exists in the industry for absorbent products, such as feminine napkins and diapers, which have a high absorbency and intake rate for complex fluids, such as blood, menses, and liquid bowel movements. Additionally, it would be beneficial if a reduced amount of superabsorbent material as compared to conventional products could be utilized to achieve these goals.

### SUMMARY OF THE INVENTION

The present invention provides absorbent articles such as tissues, tampons, feminine napkins, diapers, incontinence products, surgical drapes and gowns, floor mats used during surgery, sponges and wipers for surgery, wound dressings, and the like comprising at least about 5% (based on the total weight of the superabsorbent particles) superabsorbent particles in the form of flakes, sheets, or ribbons having an average surface area to volume ratio of at least about 0.05 μm⁻¹. By providing a substantially non-spherical, flat superabsorbent material in flake, sheet, or ribbon form, as opposed to conventional spherical-like superabsorbent particles, having a high surface area to volume ratio in the absorbent product, the product exhibits significantly improved properties when contacted with a complex bodily fluid, such as plasma, blood, menses, mucus, or liquid bowel movement. The superabsorbent flakes described herein and having a high surface area to volume ratio exhibit the fluid intake speed and capacity of spherical-like superabsorbent particles of a very small diameter without the shortcomings described above.

In addition to having a specified surface area to volume ratio, the flake superabsorbent particles contained in the absorbent products of the present invention also have an average thickness of from about 10 micrometers to about 30 micrometers, and an average particle size distribution of from about 100 micrometers to about 850 micrometers. Flake superabsorbent particles having these dimensions exhibit excellent intake speed and capacity in absorbent articles when contacted with complex bodily fluids.

Briefly, therefore, the present invention is directed to an absorbent article for absorbing a complex fluid comprising an absorbent structure and superabsorbent particles. At least about 5% (by total weight of the superabsorbent particles) of the superabsorbent particles present in the article are in flake form and have an average surface area to volume ratio of at least about 0.05 μm⁻¹.

The present invention is further directed to a feminine napkin comprising an absorbent structure and superabsorbent particles. At least about 5% (by total weight of the superabsorbent particles) of the superabsorbent particles are in flake form and have an average surface area to volume ratio of at least about 0.05 μm⁻¹ and a thickness of no more than about 40 micrometers.

The present invention is further directed to a method for absorbing blood or menses comprising contacting the blood or menses with an absorbent article comprising superabsorbent particles. At least about 5% (by total weight of the superabsorbent particles) of the superabsorbent particles are in flake form and have an average surface area to volume ratio of at least about 0.05 μm⁻¹ and a thickness of no more than about 40 micrometers.

Other features and advantages of this invention will be in part apparent and in part pointed out hereinafter.

### BRIEF DESCRIPTION OF THE DRAWINGS

FIG. 1 is a photomicrograph of granule superabsorbent particles prepared in Example 1 herein.

FIG. 2 is a photomicrograph of flake superabsorbent particles prepared in Example 1 herein.

### DETAILED DESCRIPTION OF THE PREFERRED EMBODIMENTS

In accordance with the present invention, the intake speed and capacity of absorbent articles, such as feminine napkins, is significantly improved by utilizing a superabsorbent particle having a flake, ribbon, or sheet form and having a high average surface area to volume ratio. More specifically, it has been discovered that by utilizing superabsorbent particles in flake form having an average surface area to volume ratio of at least about 0.05 μm⁻¹, the absorbent product can absorb complex bodily fluids such as plasma, blood, menses, mucus, and liquid bowel movement in an improved manner as compared to conventional absorbent products utilizing spherical-like superabsorbent particles.

Numerous absorbent products, which comprise an absorbent body or structure and utilize superabsorbent particles for absorbing liquids, can incorporate the flake-like superabsorbent materials described herein in accordance with the present invention. For example, suitable medical absorbent products include surgical drapes and gowns, floor mats used during surgery, sponges or wipers used during surgery, and wound dressings. Suitable absorbent products also include tampons, feminine napkins, interlabial devices, diapers, incontinence garments, training pants, tissue products and the like. A particularly desirable absorbent product comprising the superabsorbent particles described herein includes feminine napkins utilized for absorbing menses.

Absorbent products designed to absorb complex bodily fluids are particularly suitable for use with the flake-like superabsorbent particles described herein. As used herein, the term “complex bodily fluid” or “complex fluid” means bodily fluids secreted or excreted by the body which comprise numerous components and have a viscosity typically higher than urine. Some complex fluids include, for example, plasma, blood, menses, mucus, liquid and semi-liquid bowel movements, and the like.

The superabsorbent particles described herein for use in combination with an absorbent product are in flake, ribbon, or sheet-like form, as opposed to conventional superabsorbent particles which are typically spherical-like or chunk-like in form; that is, the superabsorbent particles described herein are substantially flat or planar in form, and do not have nearly equal size in length, width, and thickness in the x, y, and z directions. Although commonly referred to herein as “flake superabsorbents” it should be understood that this not only refers to flake superabsorbents, but also ribbon and sheet-like superabsorbent materials. As noted above, superabsorbent polymers, also commonly referred to as ionic hydrogels or ionic hydrocolloids, are typically cross-linked ionic polymers.

Superabsorbent polymers in flake-like form for incorporation into one or more of the various products described herein can be prepared by two separate manufacturing approaches described herein. The first approach utilizes a monomer solution, and is referred to herein as the “monomer-gel extrusion method”. The second method utilizes a pre-polymer solution, and is referred to herein as the “pre-polymer method”.

Monomer-Gel Extrusion Method

When a monomer solution is utilized to prepare the superabsorbent flakes, the monomer is used in combination with a neutralization agent, a crosslinking agent, an initiator and water. An aqueous monomer solution is first prepared by combining the monomer, neutralization agent, crosslinking agent, and water while stirring. After a homogeneous mixture is created, the initiator is added to the mixture to initialize the polymerization reaction, which results in the formation of a crosslinked superabsorbent gel. The crosslinked superabsorbent gel, which contains water, is then extruded through dies to form ribbons or noodles of superabsorbent polymer. Typically, the extrusion gap has a thickness of from about 50 micrometers to about 10,000 micrometers, suitably from about 100 micrometers to about 5000 micrometers, and more suitably from about 200 micrometers to about 2000 micrometers to produce the ribbons or noodles of superabsorbent gel.

After the extrusion is complete, the gel is dried in an oven for a period of from about 0.1 hours to about 24 hours, suitably from about 2 hours to about 12 hours at a temperature of from about 60° C. to about 200° C., suitably from about 70° C. to about 150° C., and more suitably from about 80° C. to about 100° C. After the drying is complete, the resulting dried ribbon-like or noodle-like superabsorbent is ground into flakes and sieved to recover the flake superabsorbent in a suitable particle size distribution range. With this approach, surface area to volume ratio is determined by two factors; thickness of the extrusion dies (the thicker the dies the thicker the resulting flake superabsorbent) and the concentration of the monomer solution (the higher the concentration, the thicker the flake superabsorbent). No subsequent heat treatment is required as the superabsorbent flakes are already crosslinked. However, known surface crosslinking and surface treatment agents widely used in the current commercial superabsorbent industry can be readily applied to the surface of the superabsorbent flakes.

Suitable monomers for preparing the superabsorbent flakes of the present invention include, but are not limited to ethylenically unsaturated monomers having at least one pendant group exhibiting either acidic, anionic, basic, or cationic functionality. Such functional groups include, but are not limited to, carboxylic acid groups, carboxyl groups, sulfonic acid groups, sulphate groups, sulfite groups, phosphoric acid groups, phosphonate groups, amino groups, imine groups, amide groups, and quaternary ammonium groups. Examples include acrylic acid, sodium acrylate, vinyl acetic acid, methacrylic acid, methacrylate, 2-acrylamido-2-methylpropanesulfonic acid, vinyl sulfonic acid, vinyl phosphoric acid, vinyl amine, allyamine, diallyldimethyl ammonium chloride, ethylene imine, acrylamide, methacrylamide, 3-amino-1-propanol vinyl ether, 3-acrylamidopropyl trimethyl ammonium chloride, hydroxyethyl acrylate, dimethylaminoalkyl(meth)-acrylate, ethoxylated (meth)-acrylates, dimethylaminopropylacrylamide or acrylamidopropyltrimethylammonium chloride. Suitable acidic or anionic monomers may comprise carboxylic acid or carboxyl groups, and suitable basic or cationic monomer may comprise amino, imine or quaternary ammonium groups.

The monomer or monomers are dissolved in a solvent at a concentration ranging from about 1 wt. % to about 60 wt. %. In order to achieve high absorbency, the monomer is suitably neutralized. An acidic monomer has to be neutralized by a base to form an anionic monomer, while a basic monomer has to be neutralized by an acid to form a cationic monomer.

Suitable neutralization agents for the monomer-gel extrusion method include hydroxides, salts, and acids. For example, when an acidic monomer is selected, suitable neutralization agents include sodium hydroxide, potassium hydroxide, ammonium hydroxide, sodium hydrogen carbonate, sodium carbonate, potassium carbonate, and sodium acetate. In another example, when a basic monomer is selected, suitable neutralization agents include hydrogen chloride, acetic acid, formic acid, ammonium chloride, sulfonic acid, and phosphoric acid.

Suitable initiators for the monomer-gel extrusion method typically include azo or peroxide/persulfate compounds, redox systems, or UV initiators (sensitizers). Radiation may also be used for initiation of the free-radical polymerization.

As noted above, a crosslinking agent is utilized in the production of the flake superabsorbent particles via the monomer-gel extrusion process to make the resulting superabsorbent particles insoluble in aqueous solution. Suitable crosslinking agents for this process include reactive crosslinking agents, which participate directly in the polymerization reaction. Suitable reactive crosslinking agents include at least two ethylenically unsaturated functional groups which are able to form a bridge between two macromolecular chains or one ethylenically unsaturated functional group and one functional group which is reactive towards the pendant groups of the monomer. Examples of suitable reactive crosslinking agents include methylene-bis-acrylamide, dimethacrylate, trimethacrylate, allyl methacrylate, methacrylate ester of glycerol and pentaerythritol, diacrylate ester of trimethylolpropane, allyl esters of phosphoric acid, and alkoxylated allyl methacrylate.

The crosslinking agent concentration utilized in the manufacture of the flake superabsorbent particles affects both the absorbency and the absorbency under load characteristics of the resulting flakes. Specifically, the higher the concentration of crosslinking agent utilized, the lower the overall absorbency of the superabsorbent flake, but the higher the swollen gel stiffness. This is due to an increased amount of polymeric internal crosslinking which reduces absorbent capacity but improves retention characteristics. Typically, the concentration of crosslinking agent is from about 0.01% (by total weight of the dry polymer) to about 20% (by total weight of the dry polymer), suitably from about 0.1% (by total weight of the dry polymer) to about 10% (by total weight of the dry polymer), and more suitably from about 0.5% (by total weight of the dry polymer) to about 5% (by total weight of the dry polymer). It should be noted that crosslinking agents can be utilized both in the bulk of the polymer and on the surface of the prepared flakes. Crosslinking density in the bulk and on the surface can be either the same or different in order to achieve optimized absorbency performance.

Pre-Polymer Method

When superabsorbent flakes are prepared from a water-soluble pre-polymer, the polymer is dissolved in water at the desired concentration level. The concentration of the polymer directly affects the resulting thickness, and hence the resulting surface area to volume ratio of the flake superabsorbent particles. The higher the concentration of polymer, the thicker the resulting flake superabsorbent particles. After dissolution of the polymer agent in the water, the crosslinking agent is added to the aqueous solution at the desired level based on the total dry weight of the polymer, and the resulting solution stirred vigorously for a time period sufficient to achieve a uniform solution.

After a uniform solution has been achieved, the solution is cast onto a flat surface to achieve a uniform thickness of the solution. Typically, the solution will be cast to a thickness of from about 1 millimeter to about 10 millimeters. After the casting is complete, the solution film is dried in an oven for a period of from about 4 hours to about 24 hours, suitably from about 6 hours to about 12 hours at a temperature of from about 60° C. to about 100° C., suitably from about 70° C. to about 100° C., and more suitably from about 80° C. to about 100° C. to produce a film of dried polymer having a thickness of from about 10 micrometers to about 100 micrometers, suitably from about 10 micrometers to about 50 micrometers, and more suitably from about 10 micrometers to about 30 micrometers.

After the drying is complete, the resulting dried ribbon-like or noodle-like superabsorbent is ground into flakes and sieved to recover the flake superabsorbent in a suitable particle size distribution range. Once the particles have been recovered, the flakes are heat treated to impart crosslinking and insolubility in water at a temperature of from about 80° C. to about 250° C., suitably from about 100° C. to about 200° C., more suitably from about 120° C. to about 150° C. for a time period of from about 1 second to about 10 hours, suitably from about 1 minute to about 5 hours, and more suitably from about 10 minutes to about 2 hours.

Suitable polymers for preparing the superabsorbent flakes of the present invention include, but are not limited to, any water soluble polyelectrolytes or polymers which are capable of being converted into polyelectrolytes through an in-situ neutralization or ion exchanging process. Examples of such polymers include (1) anionic polymers and their respective polymers in acid forms; (2) cationic polymers and their respective polymers in base forms; and (3) mixtures of anionic polymers and their respective polymers in acid forms and cationic polymers and their respective polymers in acid forms.

Suitable anionic (or acidic), water-swellable, water-insoluble superabsorbent polymers include functional groups that are capable of generating or being converted to anions. Such functional groups include, but are not limited to, carboxyl groups, sulfonic groups, sulphate groups, sulfite groups, and phosphate groups. In one embodiment, the functional groups are carboxyl groups. Generally, the functional groups are pendant groups and attached to a linear base polymer. Suitable base polymers include polyacrylates, polyacrylamides, polyvinyl alcohols, ethylene maleic anhydride copolymer, polyvinyl ethers, polyacrylamido methylpropane sulfonic acid, polyacrylic acids, polyvinylpyrrolidones, polyvinyl morpholines, and copolymers thereof. Natural based polysaccharide polymers may also be used, including carboxymethyl celluloses, carboxymethyl starches, acrylic grafted celluloses, hydrolyzed starch grafted polyacrylonitriles, and copolymers thereof. Synthetic polypeptides can also be used, such as polyaspartic acid and polyglutamic acid.

Suitable cationic (or basic) water-swellable, water-insoluble polymers include functional groups that are capable of generating or being converted to cations. Such functional groups include, but are not limited to, quaternary ammonium groups, primary, secondary, or tertiary amino groups, imino groups, imido groups, and amido groups. In one embodiment, the functional groups are quaternary ammonium groups and primary amino groups. Generally, the functional groups are pendant groups and attached to a linear base polymer. Suitable base polymers include polyamines, polyethyleneimines, polyacrylamides, polyvinylamines, polydiallyl dimethyl ammonium hydroxide, polyquaternary ammoniums, and copolymers thereof. Natural based polysaccharide polymers may also be used, including chitin and chitosan. Synthetic polypeptides can also be used, such as polyasparagines, polyglutamines, polylysines, and polyarginines.

When an acidic water-swellable, water-insoluble polymer is used as the superabsorbent material, suitably at least about 50 molar percent, or at least about 70 molar percent, or at least about 90 molar percent, or substantially about 100 molar percent, of the acidic polymer's acidic functional groups are in free acid form. In order to achieve a high absorbency, a basic neutralization agent is used, which can be either a water-swellable, water insoluble polymer or a non-polymer based organic or inorganic compound. Examples of suitable basic neutralization agents include, but are not limited to, polymeric basic materials such as polyamines, polyimines, polyamides, polyquaternary ammoniums, chitins, chitosans, polyasparagines, polyglutamines, polylysines, and polyarginines; organic basic materials such as organic salts, for example, sodium-citrate, and aliphatic and aromatic amines, imines, and amides; and inorganic bases such as metallic oxides, for example, calcium oxides; hydroxides, for example, barium hydroxide; salts such as sodium carbonate and sodium bicarbonate; and combinations of any of these.

When a basic, water-swellable, water-insoluble polymer is used, typically at least about 50 molar percent, or at least about 70 molar percent, or at least about 90 molar percent, or substantially about 100 molar percent of the basic polymer functional groups are in free base form. In order to achieve a high absorbency, an acidic neutralization agent is used, which can be either a water-swellable, water-insoluble polymer or a non-polymer based organic or inorganic compound. Examples of suitable acidic neutralization agents, include, but are not limited to, polymeric acidic materials such as polyacrylic acid, polymaleic acid, carboxymethyl cellulose, alginic acid, polyaspartic acid, and polyglutamic acid; organic acidic material such as aliphatic and aromatic acids, for example, citric acid, glutamic acid or aspartic acid; and inorganic acids such as metallic oxides, for example, aluminum oxide; and salts such as iron chloride, calcium chloride and zinc chloride; and combinations of any of these.

Polymers suitable for use in producing the flake superabsorbent particles described herein typically have a molecular weight of from about 10,000 to about 10,000,000, suitably from about 100,000 to about 10,000,000, and more suitably from about 500,000 to about 10,000,000. The molecular weight of the polymer utilized in the preparation of the flake superabsorbent particles directly affects the absorbency characteristics of the resulting flake superabsorbent particles. Typically, the higher the molecular weight of the polymeric material, the higher the resulting absorbency of the flake superabsorbent particles produced therefrom.

The polymeric materials have a higher degree of absorbency when they are highly charged or have a higher degree of charge density along the polymer backbone. As such, the degree of neutralization of the polymer can be from about 30% to about 100%, suitably from about 40% to about 100%, and more suitably from about 50% to about 100% to ensure sufficient charge density is created along the polymeric backbone to achieve suitable absorbency.

The polymer is utilized in the superabsorbent particle manufacturing process at a concentration level sufficient to produce flake superabsorbent particles having the desired dimensional and absorbency characteristics. Typically, the lower the concentration of polymer utilized, the thinner the resulting flakes, and typically the larger the surface area to volume ratio of the resulting flakes. Specifically, the concentration of polymer utilized in the manufacturing process may be from about 0.1% (by weight) to about 30% (by weight), suitably from about 0.2% (by weight) to about 20% (by weight), and more suitably from about 0.5% (by weight) to about 10% (by weight).

A latent crosslinking agent is suitable for use in the flake-like superabsorbent particle manufacturing process using a pre-polymer. Latent crosslinking agents are not reactive in the polymerization step of the superabsorbent manufacturing process, but are reactive after the polymer is shaped and a proper external condition is applied, such as heat, light, radiation, humidity, pressure, etc. Through the application of heat, for example, the latent crosslinking agent is activated and crosslinks the polymer making it insoluble in water. Latent crosslinking agents may be used in either the monomer or the pre-polymer approach to making superabsorbent particles. A suitable post treatment condition to induce intermolecular crosslinking includes using heat treatment to a temperature above about 60° C., and suitably above 100° C.

Latent crosslinking agents suitable for use in the preparation of superabsorbent polymers in accordance with the present invention are typically water-soluble crosslinking agents. A suitable latent crosslinking agent is an organic compound having at least two functional groups or functionalities capable of reacting with any carboxyl, carboxylic acid, amino, or hydroxyl groups on the polymer backbone. Examples of suitable latent crosslinking agents for use when the polymer is an anionic polymer include, but are not limited to, diamines, polyamines, diols, polyols, dicarboxylic acids, polycarboxylic acids, and polyoxides. Another suitable latent crosslinking agent comprises a metal ion with more than two positive charges, such as Al³⁺, Fe³⁺, Ce³⁺, Ce⁴⁺, Ti⁴⁺, Zr⁴⁺, and Cr³⁺. When the polymer is a cationic polymer, a suitable latent non-polymerizable crosslinking agent is a polyanionic material such as sodium polyacrylate, carboxymethyl cellulose, or polyphosphate.

The crosslinking agent concentration utilized in the manufacture of the flake superabsorbent particles affects both the absorbency and the absorbency under load characteristics of the resulting flakes. Specifically, the higher the concentration of crosslinking agent utilized, the lower the overall absorbency of the superabsorbent flake, but the higher the swollen gel stiffness. This is due to an increased amount of polymeric internal crosslinking which reduces absorbent capacity but improves retention characteristics. Typically, the concentration of crosslinking agent is from about 0.01% (by total weight of the dry polymer) to about 20% (by total weight of the dry polymer), suitably from about 0.1% (by total weight of the dry polymer) to about 10% (by total weight of the dry polymer), and more suitably from about 0.5% (by total weight of the dry polymer) to about 5% (by total weight of the dry polymer). It should be noted that crosslinking agents can be utilized both in the bulk of the polymer and on the surface of the prepared flakes. Crosslinking density in the bulk and on the surface can be either the same or different in order to achieve optimized absorbency performance.

As discussed herein, the superabsorbent particles for use in combination with an absorbent article such as a feminine napkin, are flake superabsorbent particles having a high surface area to volume ratio as compared to conventional spherical-like or chunk-like superabsorbent particles which have lower surface area to volume ratios. As is known, a sphere minimizes the surface area to volume ratio of a material such as a superabsorbent polymeric material. In contrast, a film of very small thickness maximizes the surface area to volume ratio of a superabsorbent polymeric material. As such by producing superabsorbent particles in flake form, a higher surface area per mass is generated as compared to the same polymer in near-spherical particulate form. Because of the higher surface area to volume ratio, flake-like superabsorbent particles have the advantages of intake speed and capacity similar to very small spherical-like superabsorbent particles, without the shortcomings of the small spheres as discussed above, which significantly limit their usefulness. The high surface area to volume ratio of the flake superabsorbent particle is suitable when used for absorbing complex fluids as these fluids are typically viscous, mucus-type fluids which quickly reduce the ability of a superabsorbent particle to absorb to capacity. Because of the high surface area and low volume of the flake superabsorbent particles, such particles are highly suited for absorbing complex fluids.

Typically, the absorbent products described herein, which incorporate superabsorbent particles, will comprise at least about 5% (by total weight of the superabsorbent particles) flake superabsorbent particles for improving the rate of intake and capacity of the absorbent article for complex fluids. Suitably, the absorbent product will comprise at least about 10% (by total weight of the superabsorbent particles) flake superabsorbent, more suitably at least about 15% (by total weight of the superabsorbent particle), and still more suitably at least about 20% (by total weight of the superabsorbent particles), or even about 25% (by total weight of the superabsorbent particles), about 35% (by total weight of the superabsorbent particles), about 50% (by total weight of the superabsorbent particles), about 70% (by total weight of the superabsorbent particles), about 90% (by total weight of the superabsorbent particles), or even about 100% (by total weight of the superabsorbent particles) flake superabsorbent.

Furthermore, the flake superabsorbent particles as described herein and utilized in an absorbent article have an average surface area to volume ratio greater than the average surface area to volume ratio of spherical-like or chunk-like superabsorbent particles. Because the superabsorbent particles produced during manufacturing are not identical in size, the surface area to volume ratios reported herein are reported as average surface area to volume ratios. As will be recognized by one skilled in the art based on the disclosure herein, some of the particles will have a higher surface area to volume ratio, and some will have a lower surface area to volume ratio as compared to the average surface area to volume ratio. Specifically, the average surface area to volume ratio of the flake-like superabsorbent particles described herein is suitably at least about 0.05 μm⁻¹, and typically at least about 0.10 μm⁻¹. Suitably the average surface area to volume ratio of the flake superabsorbent particles is at least about 0.15 μm⁻¹, or even 0.20 μm⁻¹. Such an average surface area to volume ratio allows the superabsorbent materials to absorb complex bodily fluids efficiently and in an improved manner as compared to conventional superabsorbent polymer particles which have an average surface area to volume ratio less than 0.05 μm¹.

Flake superabsorbent particles present in the absorbent products with an average thickness of no more than about 40 micrometers significantly improve the overall performance of the superabsorbent particles; that is, the average thickness of all of the flake superabsorbent particles present in the absorbent product is suitably no more than about 40 micrometers. Such a thickness allows the flake superabsorbent particles to achieve the desired average surface area to volume ratio. Typically, the flake superabsorbent particles will have an average thickness of from about 10 micrometers to about 30 micrometers, and suitably from about 15 micrometers to about 25 micrometers.

Generally, the particle size of the flake superabsorbent particles utilized in combination with the absorbent products can vary widely within the scope of the present invention. Suitably, the flake superabsorbent particles can have an overall average particle size distribution in the absorbent product of from about 100 micrometers to about 850 micrometers, and more particularly an average particle size of from about 100 micrometers to about 400 micrometers. In one specific embodiment, the average particle size may be from about 100 micrometers to about 200 micrometers or even about 106 micrometers to about 112 micrometers.

### EXAMPLE 1

In this Example, superabsorbent particles in flake form and superabsorbent particles in granular (spherical-like) form were manufactured and recovered using a pre-polymer process as described herein. The recovered flake superabsorbent particles and granular superabsorbent particles had a particle size distribution of less than about 850 micrometers.

Into 1000 grams of distilled water was added and dissolved 20 grams of carboxymethyl cellulose linear polymer, available from Hercules Inc. (Wilmington Del.) (CMC-7H4F). To the homogeneous aqueous solution was added 1.5 grams of BACOTE solution containing 40% ammonium zirconium carbonate (Magnesium Elektron, Inc., Flemington, N.J.) as a latent crosslinking agent. The resulting solution was stirred vigorously in a one gallon Kitchen Aid mixer for 90 minutes to achieve a high degree of uniformity.

After the mixing was complete and a high degree of uniformity achieved, one half of the solution was transferred into a 1000 milliliter glass beaker for preparation of the granular superabsorbent particles. The other half of the solution (used for preparing the flake superabsorbent particles) was further diluted by adding another 1000 grams of distilled water and the resulting solution stirred for about 10 minutes to mix the components. The resulting stirred solution was then cast into a flat surface pan having a dimension of about 20 inches wide by about 20 inches long and being about 1 inch deep to form a uniform thin solution in the pan. Both the beaker and the pan were then introduced into an oven at a temperature of about 25° C. for about 48 hours. At the end of about 48 hours, the temperature of the oven was raised to about 80° C. for about 5 hours after which the beaker and pan were removed. The dry thickness of the film produced by drying the resulting polymer in the flat surface pan was about 20 micrometers.

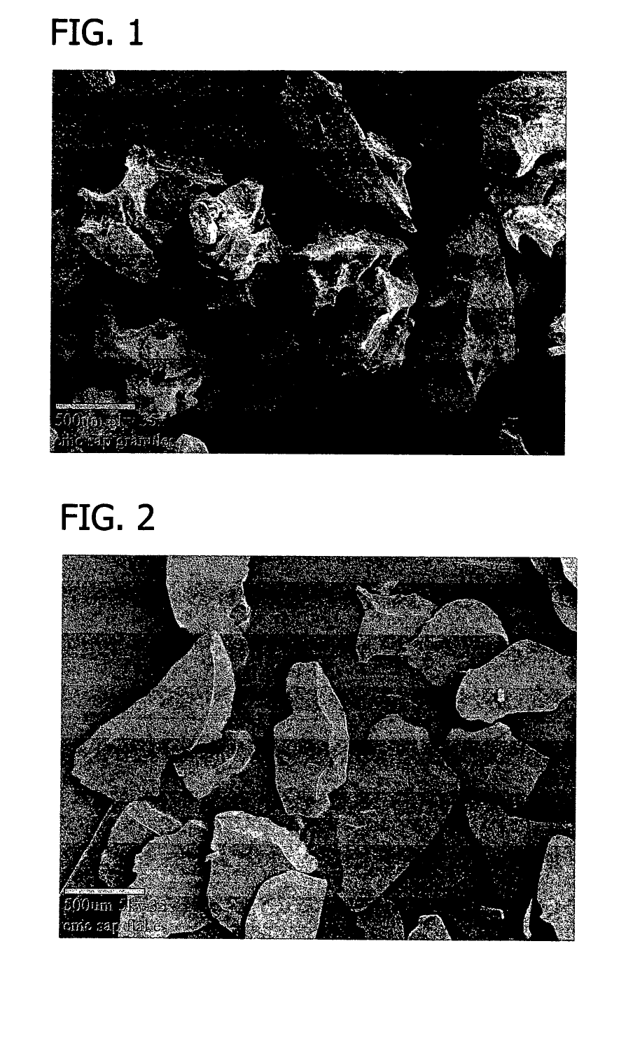

Both granular and flake dried polymer were then separately ground using an OSTERIZER 12-speed blender (Sunbeam Products, Inc., Boca Raton, Fla.). The blended granules or flakes were then sieved through a screen having an 850 micrometer opening. Agitation during sieving ensured that the width and thickness of the particles were less than 850 micrometers. For some particles, the length dimension may be been greater than 850 micrometers. After sieving, the flakes and granules were visualized by a Scanning Electron Microscope (JSM-840 from J.E.O.L., Peabody, Mass.) and photomicrographs were taken. Three dimensions of about twenty randomly selected flakes or granules were measured on the computer screen using software (SEMICAPS Genie v. 1.0 desktop imaging system manufactured by SEMICAPS, Inc., Santa Clara, Calif.) in conjunction with the scanning electron micrograph. The resulting flakes had an average surface area to volume ratio of about 0.098 μm⁻¹ and an average thickness of about 22.6 micrometers. The resulting granules had an average surface area to volume ratio of about 0.0082 μm⁻¹. The thickness of the granules was approximately that of their width. A photomicrograph of the granules is shown in FIG. 1 and a photomicrograph of the flakes is shown in FIG. 2.

### EXAMPLE 2

In this Example, bench testing was performed to evaluate the Centrifuge Retention Capacity for saline of the granule and flake superabsorbent particles prepared in Example 1.

The Centrifuge Retention Capacity Test measures the ability of superabsorbent gel particles (granules and flakes in this Example) to retain a certain liquid (i.e., saline or blood) therein after being saturated and subjected to centrifugation under a controlled set of conditions. The resultant retention capacity is stated as grams of liquid retained per gram weight of the sample (g/g). The sample to be tested is prepared from superabsorbent particles which are prescreened through a U.S. standard 30 mesh screen and retained on a U.S. standard 50 mesh screen. As a result, the sample comprises particles sized in the range of from about 300 micrometers to about 600 micrometers. The particles can be prescreened manually or mechanically and are stored in a sealed airtight container until testing.

The retention capacity was measured by placing 0.2±0.005 grams of the prescreened sample into a water-permeable bag (heat-sealable tea bag material 1234T available from Dexter Corporation, Windsor Locks, Conn.) which contains the sample while allowing a test solution (0.9 weight percent sodium chloride in distilled water) to be freely absorbed by the sample. The bag was formed by folding a 5 inch by 3 inch sample of the bag material in half and heat-sealing two of the open edges to form a 2.5 inch by 3 inch rectangular pouch. The heat seals were about 0.25 inches inside the edge of the material. After the sample was placed in the pouch, the remaining open edge of the pouch was heat sealed. The samples were prepared in triplicate. The filled bags were tested within no more than three minutes of preparation.

Once filled, the bags were placed between two TEFLON coated fiberglass screens having 3 inch openings (Taconic Plastics, Inc., Petersburg, N.Y.) and submerged in a pan of the test solution at 23° C., making sure that the screens were held down until the bags were completely wetted. After wetting, the samples remained in the solution for about 30 minutes and then removed from the solution and temporarily laid on a non-absorbent flat surface.

The wet bags were then placed into the basket of a Heraeus LaboFuge 400 having a water collection basket, a digital rpm gauge, and a machined drainage basket adapted to hold and drain the bag samples. The centrifuge was capable of subjecting the samples to a g-force of about 350. The bags were then centrifuged at about 1600 rpm for about 3 minutes. The samples were tested at about 23° C. and about 50 percent relative humidity. The bags were then removed and weighed along with empty bags used as controls. The amount of solution retained by the sample, taking into account the solution retained by the bag itself, is the centrifuge retention capacity of the sample, expressed as grams of fluid per gram of sample. The retention capacity was calculated as follows:
CRC=(W_{(sample+bag) after centrifuge}−W_{(bag) after centrifuge}−W_{dry sample})/W_{(dry sample)}

The results are shown in Table 1:

TABLE 1

| Sample # | SAP | CRC (g/g) |
|---|---|---|
| 1 | Flakes | 19.56 |
| 2 | Granules | 17.76 |

As the data in Table 1 indicate, the granule superabsorbent particles and flake superabsorbent particles had similar retention capacities for saline solution. Because saline solution is not a complex solution, it is expected that the flake superabsorbent particles and granular superabsorbent particles would have similar absorption capacity as each bag comprised about the same amount of superabsorbent particles.

### EXAMPLE 3

In this Example, bench testing was performed to evaluate the Centrifuge Retention Capacity in blood of the granule and flake superabsorbent particles prepared in Example 1.

In this Example, the granule and flake superabsorbent particles evaluated had a particle size distribution of less than about 850 micrometers. The centrifuge retention capacity was measured by first introducing 0.04 grams of the prescreened sample (see Example 2) superabsorbent particles into a plastic ring having a diameter of about 50 millimeters with a 160 micrometer mesh screen attached to the bottom. Prior to the introduction of the superabsorbent particles, the plastic ring and mesh screen were weighed. The superabsorbent particles were separated as evenly as possible over the screen at the bottom of the plastic ring. Four replicates for each type of superabsorbent particle was prepared.

Swine blood (Cocalico Biologicals, Inc., Reamstown, Pa.) was adjusted to about 35% hematocrit by dilution with swine plasma or concentration through centrifugation, as necessary. Twenty milliliters of the 35% hematocrit swine blood was introduced via syringe into a glass cup located under each plastic ring and screen containing the superabsorbent samples. Forceps were used to grasp the plastic ring and introduce it into the cup containing the swine blood such that the mesh screen and superabsorbent particles were completely submerged in the swine blood such that the superabsorbent particles could absorb blood from both the top and bottom. A cover was placed over the top of the glass cup to minimize evaporation. The superabsorbent particles were allowed to soak in the swine blood for either 30 or 90 minutes, depending upon the test.

After the superabsorbent particles had soaked in the swine blood for the desired test time, the plastic ring and mesh screen containing the superabsorbent particles were removed from the glass cup containing the blood and centrifuged in cup holders designed to keep the plastic ring and mesh screen from exposure to centrifuged blood to remove blood from the interstitial spaces among the swollen superabsorbent particles. Centrifugation was done at 1250 rpm in a Sorvall RT 6000D centrifuge (Kendro Laboratory Products, Asheville, N.C.) for three minutes. The plastic ring and mesh screen containing the superabsorbent particles were then removed from the centrifuge and weighed. The gram per gram blood uptake was calculated according to the following equation:
CRC=(W_{(sample+holder) after centrifuge}−W_{dry holder}−W_{dry sample})/W_{dry sample}

The results for both a 30 minute and a 90 minute soaking time are shown in Table 2:

TABLE 2

| Time Period | Granular Superabsorbent | Flake Superabsorbent |
|---|---|---|
| 30 minutes | 16.00 g/g | 25.96 g/g |
| 90 minutes | 21.72 g/g | 36.77 g/g |

As the data in Table 2 indicate, the flake-like superabsorbent particles having a high surface area to volume ratio absorbed a substantially increased amount of blood at both 30 minutes and 90 minutes as compared to the granular superabsorbent particles. Without being bound to a particular theory, it is believed that this is attributable to the higher surface area of the flake-like superabsorbent particles providing additional sites to deposit the complex components of blood (i.e., mucin, red blood cells, etc.). It is believed that while superabsorbents swell with water from the blood, the components left in the fluid make it even more difficult for fluid movement into the superabsorbent particle. It is believed that the flake-like superabsorbent particles may be immobilizing some of these other components, thereby allowing the fluid to be more mobile. It is further believed that the shorter diffusion path length for fluids to fully penetrate a superabsorbent particle in the shape of a flake, as compared to a chunk, leads to a higher capacity in complex fluids.

In view of the above, it will be seen that the several objects of the invention are achieved. As various changes could be made in the above-described materials without departing from the scope of the invention, it is intended that all matter contained in the above description be interpreted as illustrative and not in a limiting sense.

1. An absorbent article for absorbing a complex fluid comprising an absorbent structure and superabsorbent particles wherein at least about 5% (by total weight of the superabsorbent particles) of the superabsorbent particles are in flake form and have an average surface area to volume ratio of at least about 0.05 μm⁻¹.

2. The absorbent article as set forth in claim 1 wherein the average surface area to volume ratio of the superabsorbent particles in flake form is at least about 0.07 μm⁻¹.

3. The absorbent article as set forth in claim 1 wherein the average surface area to volume ratio of the superabsorbent particles in flake form is at least about 0.10 μm⁻¹.

4. The absorbent article as set forth in claim 1 wherein the average surface area to volume ratio of the superabsorbent particles in flake form is at least about 0.20 μm⁻¹.

5. The absorbent article as set forth in claim 1 wherein the superabsorbent particles having the average surface area to volume ratio of at least about 0.05 μm⁻¹ have an average thickness of no more than about 40 micrometers.

6. The absorbent article as set forth in claim 1 wherein the superabsorbent particles having the average surface area to volume ratio of at least about 0.05 μm⁻¹ have an average thickness of from about 10 micrometers to about 30 micrometers.

7. The absorbent article as set forth in claim 1 wherein the superabsorbent particles having the average surface area to volume ratio of at least about 0.05 μm⁻¹ have an average thickness of from about 15 micrometers to about 25 micrometers.

8. The absorbent article as set forth in claim 1 wherein at least about 10% (by total weight of the superabsorbent particles) of the superabsorbent particles have an average surface area to volume ratio of at least about 0.05 μm⁻¹.

9. The absorbent article as set forth in claim 1 wherein at least about 15% (by total weight of the superabsorbent particles) of the superabsorbent particles have an average surface area to volume ratio of at least about 0.05 μm⁻¹.

10. The absorbent article as set forth in claim 1 wherein at least about 20% (by total weight of the superabsorbent particles) of the superabsorbent particles have an average surface area to volume ratio of at least about 0.05 μm⁻¹.

11. The absorbent article as set forth in claim 1 wherein the superabsorbent particles having an average surface area to volume ratio of at least about 0.05 μm⁻¹ have a particle size distribution of from about 100 micrometers to about 850 micrometers.

12. The absorbent article as set forth in claim 1 wherein the superabsorbent particles having an average surface area to volume ratio of at least about 0.05 μm⁻¹ have an average particle size of from about 200 micrometers to about 700 micrometers.

13. The absorbent article as set forth in claim 1 wherein the superabsorbent particles having an average surface area to volume ratio of at least about 0.05 μm⁻¹ have an average particle size of from about 300 micrometers to about 600 micrometers.

14. The absorbent article as set forth in claim 1 wherein the absorbent article is selected from the group consisting of tampons, interlabial pads, feminine napkins, diapers, incontinence products, wound dressings, surgical drapes and gowns, floor mats for surgery, and sponges or wipers used during surgery.

15. A feminine napkin comprising an absorbent structure and superabsorbent particles wherein at least about 5% (by total weight of the superabsorbent particles) of the superabsorbent particles are in flake form and have an average surface area to volume ratio of at least about 0.05 μm⁻¹ and a thickness of no more than about 40 micrometers.

16. The feminine napkin as set forth in claim 15 wherein at least about 15% (by total weight of the superabsorbent particles) of the superabsorbent particles are in flake form.

17. The feminine napkin as set forth in claim 15 wherein the average surface area to volume ratio of the superabsorbent particles in flake form is about 0.10 μm⁻¹.

18. The feminine napkin as set forth in claim 15 wherein the average surface area to volume ratio of the superabsorbent particles in flake form is about 0.20 μm^{-1.}

19. The feminine napkin as set forth in claim 15 wherein the thickness is from about 10 micrometers to about 30 micrometers.

20. The feminine napkin as set forth in claim 15 wherein the thickness is from about 15 micrometers to about 25 micrometers.

21. The feminine napkin as set forth in claim 15 wherein the superabsorbent material having an average surface area to volume ratio of at least about 0.05 μm⁻¹ and a thickness of from about 10 micrometers to about 30 micrometers has an average particle size distribution of from about 100 micrometers to about 850 micrometers.

22. The feminine napkin as set forth in claim 21 wherein the average particle size distribution is from about 200 micrometers to about 700 micrometers.

23. The feminine napkin as set forth in claim 21 wherein the average particle size distribution is from about 300 micrometers to about 600 micrometers.

24. A method for absorbing blood or menses comprising contacting the blood or menses with an absorbent article comprising superabsorbent particles wherein at least about 5% (by total weight of the superabsorbent particles) of the superabsorbent particles are in flake form and have an average surface area to volume ratio of at least about 0.05 μm ⁻¹ and a thickness of no more than about 30 micrometers.

25. The method as set forth in claim 24 wherein the average surface area to volume ratio of the superabsorbent particles in flake form is at least about 0.10 μm⁻¹.

26. The method as set forth in claim 24 wherein the average surface area to volume ratio of the superabsorbent particles in flake form is at least about 0.20 μm⁻¹.

27. The method as set forth in claim 24 wherein the thickness from about 10 micrometers to about 30 micrometers.

28. The method as set forth in claim 24 wherein the thickness from about 15 micrometers to about 25 micrometers.

29. The method as set forth in claim 24 wherein the superabsorbent particles having the average surface to volume ratio of at least about 0.05 μm⁻¹ have a particle size distribution of from about 100 micrometers to about 850 micrometers.
